(19) United States
(12) Patent Application Publication
(10) Pub. No.: US 2009/0148881 A1
(43) Pub. Date: Jun. 11, 2009

(54) GEOBACILLUS THERMODENITRIFICANS AS WELL AS THE SCREENING METHOD AND THE USES THEREOF

(75) Inventors: Lei Wang, Tianjin (CN); Rulin Liu, Tianjin (CN); Lu Feng, Tianjin (CN); Fenglai Liang, Tianjin (CN)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: NANKAI UNIVERSITY, Tianjin (CN)

(21) Appl. No.: 11/719,556

(22) PCT Filed: Mar. 22, 2005
(86) PCT No.: PCT/CN05/00360

§ 371 Date: May 17, 2007

(30) Foreign Application Priority Data
Nov. 17, 2004 (CN) 200410072759.7

(51) Int. Cl. C12Q 1/68; C12N 1/20

(52) U.S. Cl. 435/29; 435/252.5

(57) ABSTRACT

The invention provides a strain of Geobacillus thermodenitrificans as well as the screening method and the uses thereof. The strain was deposited as the number CGMCC-1228 in the China General Microbiological Culture Collection Center of the China Committee of Culture Collection for Microorganisms. The strain was obtained by primary screening, re-screening, inoculation, domestication and propagation using the bacteria in the aqueous samples from the strata of oil field as the original screening strain. The strain screened in the invention belongs to the genus Geobacillus, which can tolerate a high temperature, and has good thermostability, which can be applied to the industrial production which need the condition of thermostable enzymes, such as fermentation, etc. With its ability of growing well in the oil-reservoir environment, degrading alkanes, decreasing viscosity and increasing fluidity of crude oil, the strain is able to remarkably enhance the yield of oil and improve the transporting efficiency of oil. Additionally, with its good ability of degrading oil, it can be developed for the treatment and clearing of the material such as the oil contaminated water, etc., so it can be useful in protection of the environment. This strain also has the ability of decreasing the surface tension of substances, which can be applied to the industry of biosurfactants preparation.

### 1. FIELD OF THE INVENTION

The present invention relates to bacterial strains. More particularly, the present invention relates to the strain of Geobacillus thermodenitrificans G1788 as well as the screening method and the uses thereof.

### 2. BACKGROUND TO THE INVENTION

In the application of microorganism engineering, microbially enhanced oil recovery is a rising technology of great application prospect and economic value. Almost 60-65% of the known crude oil can not be extracted after primary and secondary oil recovery in many oil fields. After being exploited by means of water flooding for several years, oil fields (e.g. some main oil fields in East China) will then enter a high water-cut stage. As a result, they have to face serious problems such as enhanced difficulty of exploitation, increasing cost, and decreasing extraction rate. Although the tertiary oil recovery, such as thermal flooding, chemistry flooding and miscible phase flooding normally used for solving these problems may be effective to some extent, it has serious disadvantages. Thermal flooding has low efficiency of heat energy. The residual period of the chemical substances infused into the strata during chemistry flooding and the miscible phase flooding is long, and the remnants will destroy the strata, pollute the environment, moreover, complicated overground equipment is needed. Microbially enhanced oil recovery is an exploitive application of bioengineering technology in oil field exploitation. Because of its advantages, such as low cost, better adaptability, simple operation, innocuous effect on strata, and no pollution this method has caught worldwide attention and become the focus and edge-cutting technology in the studies and the practices of exploitation of petroleum resources, being regarded as the fourth kind of tertiary oil recovery having specific advantages.

The microbially enhanced oil recovery is the generic name of microbe-related technologies for enhancing extraction rate of petroleum, i.e., technologies that enhance the extraction rate of oil recovery by using microbes. Generally the basic technology may be divided into two types: one is on-ground zymotechnics, and the other is underground zymotechnics, in which the oil reservoir is regarded as a huge bioreactor to allow the microbes to undergo fermentation under ground. The under-ground zymotechnics again may be divided into two types: one is to select nourishing substances suitable for the intrinsic microbes in oil, and then infuse it to the strata, so as to activate the intrinsic microbes; the other includes steps of screening suitable strains while considering the character of the oil reservoir, and infusing the strains having been cultured and fermented along with the nourishing substances into the strata. The present invention utilizes the later one to screen more suitable strains. Depending upon the way of the operation, the microbially enhanced oil recovery by infusing strains along with nourishing substances includes four methods: (1) periodically infusing microbial recovery (also known as Single Well Throughput); (2) microbial flooding; (3) selective blockage; and (4) microbial wax clearing and preventing, wherein the microbially enhanced oil recovery mainly refers to the microbial flooding in which the microbes along with nourishing substances are infused through the affusion well into destination strata, the fluidity of crude oil is then increased by the action of the microbial metabolism in the oil layers, such that the petroleum may be exploited more easily.

The mechanism of the microbially enhanced oil recovery is very complicated. Up to now, studies found that several mechanisms may expound microbially enhanced oil recovery. They are as follows: (1) microbes may decompose heavy oil to light oil or other products, thereby reducing the viscosity and increasing the fluidity of the crude oil (2) CO₂ and CH₄ gases produced by the metabolic activity of the microbes in the oil reservoir will boost the inner pressure and allow the discontinuous crude oil zones to be joined to form one piece, which may facilitate exploiting; (3) microbes may generate several kinds of surfactants (e.g. fatty acid, lipopolysaccharides (LPS), and glucan, etc.), which will then decrease the interfacial tensions between the oil and the rock and thereby improve the fluidity of oil and enhance the efficiency of water flooding; (4) the microbes' bodies and their excretive bio-polymers may block the porous and permeable rock formations selectively, so as to turn the liquid to flow to the crevice with low permeability and enhance the efficiency of water flooding and gas flooding; (5) the acid substances generated by microbes may dissolve the rock and increase the penetrability of the strata, therefore improve the seepage of the oil. Though the mechanism of the microbially enhanced oil recovery has been continuously being studied and explored, microbially enhanced oil recovery has been used extensively and has resulted in a valuable economic effect.

The key process which determines whether the microbially enhanced oil recovery will be a success is to find and use strains with excellent function. The strains must be able to acclimatize themselves to the temperature, pressure and salinity of the oil field, as well as other environmental conditions thereof. The most important condition is the temperature condition. Therefore, thermophily of the bacteria is particularly important for oil extraction in high-temperature oil field. The strain according to the present invention is a thermophilus strained screened by a specific method, which may survive in a wide range of temperature, and be adequate to grow in the high-temperature oil field environment. Degrading the alkyl hydrocarbon in crude oil thus increasing the fluidity of crude oil is an important mechanism of the microbially-enhanced oil recovery, the strain of the present invention may use petroleum as the sole carbon source, and it has an excellent capacity of degrading alkyl hydrocarbon and crude oil, and has the excellent characters required by the strains used in microbially enhanced oil recovery.

The strain has the petroleum-degrading capability, the growth and metabolism of the strain in certain petroleum-containing substances will perform a good purification function to these substances by eliminating petroleum. At present, due to the development of the petroleum industry and the petroleum pollution on people's living environment generated by recovery, transportation and production of petroleum, the petroleum-contaminated substances caused very serious environmental pollution, such as petroleum-containing sewage, the leakage of crude oil in the environment and so on. By using the strain screened according to the present invention, the petroleum in these substances may be degraded, the contaminants may be purified, and the using of the strain may play an important role in environmental protection.

The strain according to the present invention belongs to the thermophilic bacteria, and almost all of the enzymes from thermophilic bacteria are thermo-stable. The thermal stability of enzymes from thermophilic bacteria is determined by the internal structure of the enzyme protein molecule. Research shows that the primary structure of the enzyme itself plays an important role in its thermal stability. The changes of individual amino acids in some key regions of the primary structure will cause the changes in the higher order structure of the enzyme, and will slightly increase the number of hydrogen bonds, ionic bonds or hydrophobic bonds in the structure of enzyme protein, thereby will improve the thermal stability of the while molecule. The thermal stability of the enzyme synthesized by thermophilic bacteria may be changed either by changing the temperature or by changing the configuration of existing enzyme protein. The enzymes separated from thermophilic bacteria have some excellent biological characteristics, such as thermal stability, and resistance to chemical and physical denaturing agents, organic solvents, extreme pH and other unfavorable factors. These characteristics may not only be used as the basis for designing and modifying enzymes, but also may have important application value for industrial production.

The strain according to the present invention has the function of degrading petroleum excreting surfactants, and so on. These functions will cause the decrease of petroleum viscosity, and the increase of petroleum fluidity, whereas the fluidity of petroleum influences the petroleum industry greatly. Firstly, if the fluidity of petroleum is increased, it will become easy to extract during the period of petroleum exploitation, therefore the requirements on equipment and the processing of the project are reduced, the extraction cost is reduced, and the efficiency is improved. Secondly, no matter whether petroleum is transported by means of a pipeline or other transportation ways, high fluidity is an extremely advantageous characteristics. For example, in the case of transporting by means of pipeline, if the fluidity of petroleum is not ideal enough, it is necessary to raise the temperature, pressure, etc. This will result in wasting a lot of indirect transport costs. The strain according to the present invention has a remarkable effect on enhancing the fluidity of petroleum. Therefore, the use of this strain will enormously improve the efficiency of petroleum transport.

As mentioned above, the strain screened by the present invention can be applied to the petroleum exploitation industry, the petroleum transportation industry, the environmental protection industry and other related industries.

### 3. SUMMARY OF THE INVENTION

The main object of the present invention is to overcome the above-mentioned disadvantages of the known art, and to provide a strain of Geobacillus thermodenitrificans G1788, as well as the screening method and the uses thereof. The screened strain belongs to Geobacillus thermophily, which has a very good thermal stability. It may be applied to industrial productions which require the condition of thermal-stable enzyme, such as fermentation. It can use crude oil as the sole carbon source. It can grow well in an oil field environment, and has a good alkane-degrading capability. The growth and metabolism of the strain using crude oil as the nutrient source in the oil field may improve the characteristics of crude oil, by decreasing the viscosity and enhancing the fluidity of oil. So it can effectively enhance petroleum exploitation and transportation efficiency. Its perfect ability of degrading oil may be used for dealing with substances polluted by oil, such as sewage contaminated by oil. It may play an important role in environmental protection. It may play an important role in environment protection. This strain also has the function of decreasing surface tension of some substances. It thus can be used for producing surfactants.

The objects of the present invention are achieved by the technical solutions described below.

The strain of Geobacillus thermodenitrificans according to the present invention is deposited in China General Microbiological Culture Collection Center of China Committee of Culture Collection for Microorganisms having the number of CGMCC-1228.

A method for screening the strain of Geobacillus thermodenitrificans according to the present invention, wherein the strains isolated from the aqueous samples of the strata of an oil field are used as the original strains for screening, and the strain is obtained after primary screening and re-screening, inoculation, domestication, and propagation.

In the described method for screening the strain of Geobacillus thermodenitrificans, the primary screening is carried out by culturing and screening the samples with agar medium plate at 73° C. Then the samples obtained are incubated with mineral nutrition medium using crude oil as carbon resource, shaken in oil bath at 73° C., and emulsified for dispersing. As a result, the best dispersed strains are obtained. Said re-screening is conducted in such a way that, in experiments of emulsifying for dispersing and experiments of decreasing viscosity and the freezing point, selecting the strains which are dispersed and emulsified best with best viscosity and freezing point decreasing effect. Thus the best strain is obtained. Then the best strain is inoculated with the mineral nutrition medium supplemented with liquid wax at 73° C. several times to be domesticated and propagated.

The optimal culture medium for said strain of Geobacillus thermodenitrificans according to the present invention includes carbon, nitrogen and mineral nutrition sources and yeast powder; wherein said carbon source is glucose, sucrose or starch, said nitrogen source is NaNO₃, said mineral nutrition is FeSO₄, MgSO₄, Na₂HPO₄ and K₂HPO₄; the pH of said medium is 6.5-7.5.

The optimal medium for said strain of Geobacillus thermodenitrificans, wherein said carbon source is sucrose of which the amount used if 0.1%, said nitrogen source is NaNO₃ of which the amount used is 0.1%-0.4%; the amount of said yeast powder used is 0.05%, the amount of said mineral nutrition used is 1.5-2 times more than that of basic medium.

The strain of Geobacillus thermodenitrificans according to the present invention may be used in oil exploration, oil hydrocarbon degradation, cleansing of material containing oil, and oil transportation field.

The strain of Geobacillus thermodenitrificans according to the present invention may be used in industrial production that requiring thermo-stable enzyme.

The strain of Geobacillus thermodenitrificans according to the present invention may be used in industry of biosurfactant preparation.

The uses of said strain of Geobacillus thermodenitrificans, wherein said materials containing oil is the sewage contaminated by oil, etc.

The uses of said strain of Geobacillus thermodenitrificans above-mentioned, wherein the industrial production requiring thermo-stable enzyme is fermentation industrial production.

### 4. BRIEF DESCRIPTION OF THE DRAWINGS

FIG. 1 schematically shows the evolution tree of 16SrRNA.

FIG. 2 schematically shows the evolution tree of Housekeeping gene araA.

FIG. 3 schematically shows the evolution tree of Housekeeping gene Mdh.

FIG. 4 schematically shows the evolution tree of Housekeeping gene recN.

FIG. 5 schematically shows the growth curve of the screened strain using oil as the sole carbon source.

FIG. 6 shows the schematic curve representation of the degradation status at respective stages of the fermentation of C₁₂ alkyl hydrocarbon performed by the screened strain of the present invention.

FIG. 7 shows the schematic curve representation of the degradation status at respective stages of the fermentation of C₁₇ alkyl hydrocarbon performed by the screened strain of the present invention.

FIG. 8 shows the schematic curve representation of the degradation status at respective stages of the fermentation of C₁₉ alkyl hydrocarbon performed by the screened strain of the present invention.

FIG. 9 shows the schematic curve representation of the degradation status at respective stage of the fermentation of C₂₅ alkyl hydrocarbon performed by the screened strain of the present invention.

FIG. 10 shows the schematic curve representation of the degradation status at respective stages of the fermentation of C₃₅ alkyl hydrocarbon performed by the screened strain of the present invention.

FIG. 11 shows the schematic curve representation of the degradation status at respective stages of the fermentation of C₄₆ alkyl hydrocarbon performed by the screened strain of the present invention.

FIG. 12 shows the schematic curve representation of the degradation status of the degradation of the oil performed by the screened strain of the present invention.

FIG. 13 shows the schematic representation of the surface tension result of the present invention.

FIG. 14 shows the flow chart of the process of extracting surfactants according to the present invention.

### 5. DETAILED DESCRIPTION OF THE PREFERRED EMBODIMENTS

### 1) Name of the Microorganism and its Culture Status

Geobacillus thermodenitrificans G1788 has been deposited in China General Microbiological Culture Collection Center (CGMCC). Its depositing number is CGMCC-1228.

### 2) Characteristics and Identification of the Strain

(1) Phenotypic, physiological and biochemical characteristics of the strain of G1788. The strain of G1788 were identified according to the “Manual of systemic determination of bacteriology, Beijing: Science press, 2001.2, ISBN: 7-03-008460-8, Dong Xiuzhu, Cai Miaoying et al.” and “Bergey's Manual of Determinative Bacteriology, 9^{th} edition”, its characteristics are shown in the following table 1:

TABLE 1
Phenotypic and physiological characteristics of strain G1788

| Item | G1788 |
|---|---|
| Diameter > 1.0μm | − |
| Endospore rotundity | Some are roduntidy, some are not |
| Cyst bulge | + |
| Catalase | + |
| V—P Assay | − |
| V—P culture pH <6 | + |
| pH >7 | − |
| Hydrolysis: Casein | − |
| Gelatin | + |
| Starch | + |
| Utilization of: Citrate | No growth |
| Tyrosine hydrolysis | − |
| Phenylalanine | − |
| Egg-Yolk lecithase | − |
| Deoxidation of nitrate | + |
| Item | G1788 |
| Indole production | − |
| Requiring NaCl and KCL | − |
| Requiring allantion and urea salt | − |
| Growth pH 6.8: LB | + |
| 5.7 LB | − |
| Growth NaCl: 2% | − |
| 5% | − |
| 7% | − |
| 10% | − |
| Temperature for growth: 5° C. | − |
| 10° C. | − |
| 30° C. | − |
| 40° C. | W |
| 50° C. | + |
| 55° C. | + |
| 65° C. | + |

Note:
“+” positive; “−” negative; “W” weak reaction.

The cell of strain G1788 is shaped like straight rods, 0.6˜1.0×3.1˜6.5 μm in size, Gram-positive, with peripheral flagellum, motile and has ellipse or swollen endospore on or near the end. The colonies thereof are big and dry, having cockled edge. It is catalase-positive, and may hydrolyze starch and gelatin hydrolysis. It also may deoxidize nitrate and nitrite, but cannot hydrolyze casein. Other characteristics are shown in table 1.

(2) Carbohydrates Fermentation Tests of Strain G1788

TABLE 2
Results from Test of G1788 in API 50
CHB/E Carbohydrates Culture Medium

| Tube | Substrate | Result |
|---|---|---|
| 0 | Control (0) | − |
| 1 | Glycerin (GLY) | + |
| 2 | Erythrose | − |
| 3 | D-Arabinose (DARA) | − |
| 4 | L-Arabinose (LARA) | + |
| 5 | Ribose (RIB) | + |
| 6 | D-Xylose (DXYL) | + |
| 7 | L-Xylose (LXYL) | − |
| 8 | Adonitol (ADO) | − |
| 9 | β-methyl-D-Xylopyranosid (MDX) | − |
| 10 | galactose (GAL) | − |
| 11 | Glucose (GLU) | + |
| 12 | Fructose (FRU) | + |
| 13 | Mannose (MNE) | + |
| 14 | Sorbose (SBE) | − |
| 15 | Rhamnose (RHA) | − |
| 16 | Dulcitol (DUL) | − |
| 17 | Inositol (INO) | W |
| 18 | Mannitol (MAN) | + |
| 19 | Sorbitol (SOR) | |
| 20 | α-methyl-D-Mannaside (MDM) | − |
| 21 | α-methyl-D-Glycoside (MDG) | − |
| 22 | N-acetyl-Glucosamine (NAG) | W |
| 23 | Amygdalin (AMY) | − |
| 24 | Arbutin (ARB) | − |
| 25 | Esculin (ESC) | + |
| 26 | Salicoside | − |
| 27 | Cellobiose (GEL) | + |
| 28 | Maltose (MAL) | + |
| 29 | Lactose (LAC) | − |
| 30 | Melibiose (MEL) | + |
| 31 | Sucrose (SAC) | + |
| 32 | Fucose (TRE) | + |
| 33 | Inulin (INU) | − |
| 34 | Melezitose (MLZ) | + |
| 35 | Raffinose (RAF) | − |
| 36 | Starch (AMD) | + |
| 37 | Glycogen (GLYG) | − |
| 38 | Xylitol (XLT) | − |
| 39 | Gentiobiose (GEN) | − |
| 40 | D-Turanose BioChemica (TUR) | + |
| 41 | D-Lyxouranose (LYX) | − |
| 42 | D-Tagatose (TAG) | − |
| 43 | D-fucose (DFUC) | − |
| 44 | L-fucose (LFUC) | − |
| 45 | D-Arabinitol (DARL) | − |
| 46 | L-Arabinitol (LARL) | − |
| 47 | Gluconate (GNT) | − |
| 48 | 2-Keto-Gluconate (2 KG) | − |
| 49 | 2-Keto-Gluconate (5 KG) | − |

“+” positive;
“−” negative;
“W” weak reaction.

Growth and acidification of carbohydrates tests are performed by using API 50 CHB/E carbohydrates culture medium and test strips (BioMérieux, Marcyl' étoile, France). Strain G1788 is inoculated according to the manufacturer's instruction. If the carbohydrates are ferments to produce acid, pH will decrease, that will be represented by the changed color of indicator. The test results are shown in table 2. Strain G1788 can utilize glycerin, ribose, L-arabinose, D-xylose, glucose, fructose, mannose, mannitol, α-methyl-D-glucoside, esculin, cellobiose, maltose, melibiose, sucrose, fucose, melezitose, starch, and D-turanose biochemical to ferment to produce acid, and can further slightly utilize inositol, N-acetyl-glucosamine to produce a small amount of acid. G1788 can not ferment D-arabinose, rhamnose and other kinds of saccharides. David J. etc. (FEMS Microbiology Letters, 1999, 172:85˜90), Manachini etc. (Int. J. Sys. Evol. Microbiol. 2000, 50:1331˜1337) figured out that Geobacillus thermodenitrificans can hydrolyze starch, ribose, tributyrin and xylan; it can also deoxidize nitrate and nitrite to generate gas; it can ferment arabinose, fibre-disaccharide, melezitose, melibiose, and fucose to generate acid; but it can not ferment galactose and rhamnose to generate acid; its colonies grow with a feather-like edge. G1788 has the same configuration and physiological and biochemical characteristics as Geobacillus thermodenitrificans, so it belongs to Geobacillus thermodenitrificans.

(3) Classifying the bacteria by means of evolutionary analysis of the 16S rDNA and housekeeping genes

16S rDNA widely exists in procaryotic organism and eukaryote, its function is stable, and it consists of highly conserved areas and variable areas. It is known as one of the best materials for studying the relationship of system evolution. The size of 16S rDNA molecule is around 1500 bp. The information it carried can reveal the evolutionary relationship of the biosphere but also can be operated easily. Similarly, the housekeeping genes are also preferred material used for the bacteria evolutionary analysis. The 16S rDNA and housekeeping genes evolutionary analysis is a new method rising with the bio-informatics development, by comparing these conserved gene sequences with the corresponding gene sequences of the other known bacteria, the category relationship of the bacterial strain will be clearly and exactly determined.

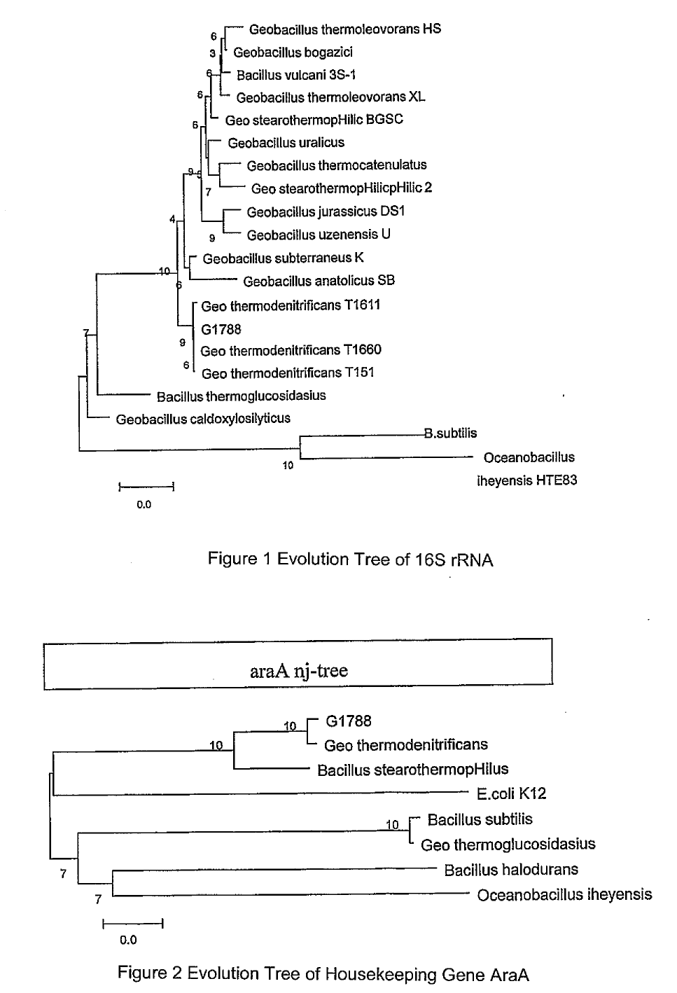

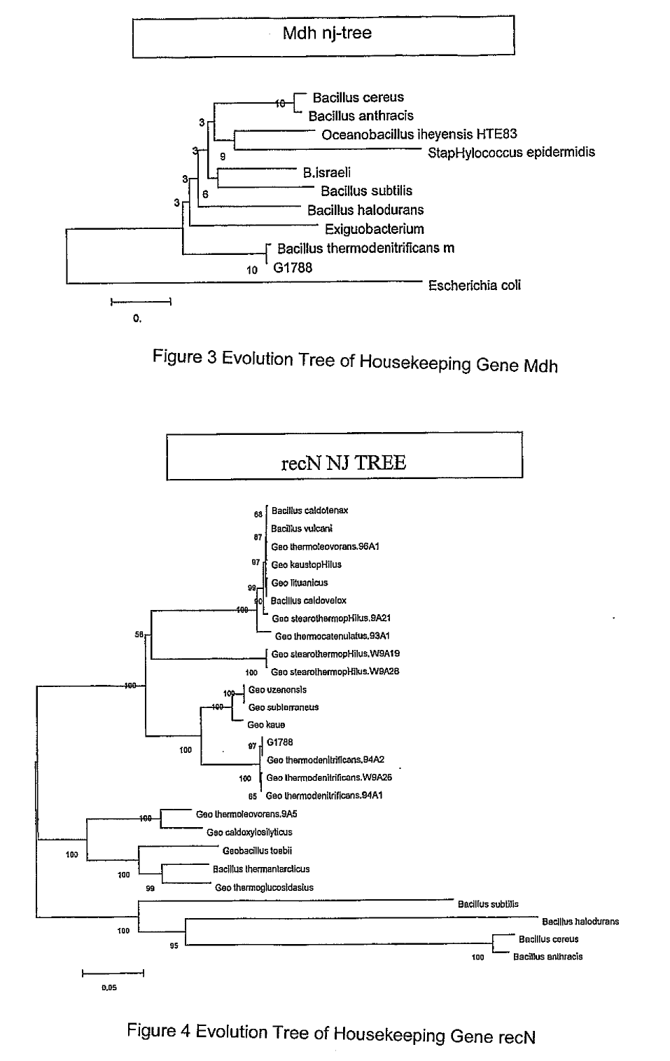

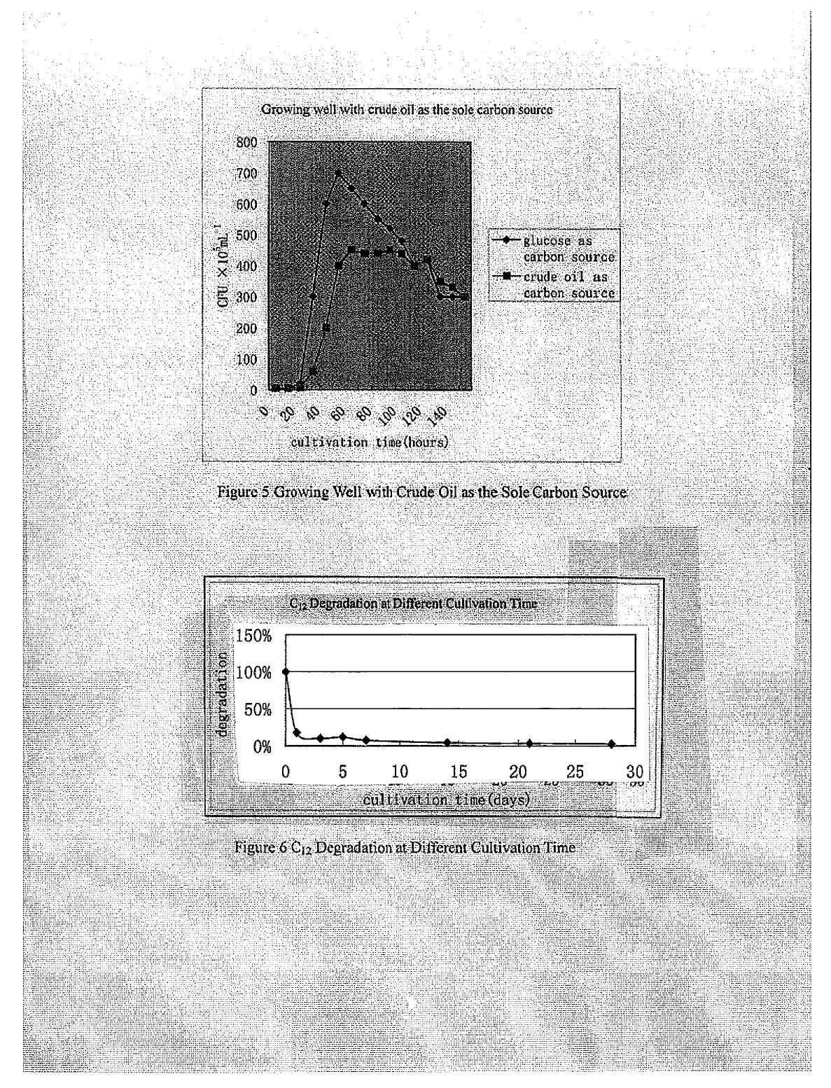

By analyzing the evolution of 16S rDNA of G1788 and housekeeping genes araA, Mdh, and recN, the 16S rDNA of G1788 and araA, Mdh, and recNare are sequenced, and are BLAST analyzed in GENEBANK and other databases, and the corresponding gene 25 sequences of several kinds of bacteria of which the race similar to G1788 are obtained. Then these gene sequences are compared by means of bio-information software, and the evolution tree is drawn according to their evolutionary relationship. Finally, the results which shared intimate relationship are adopted (referring to FIGS. 1 to 4).

The 16S rDNA of G1788 shares 99% identity of the nucleic acid sequence with that of Geobacillus thermodenitrificans T1660; while araA gene shares 95% identity of the nucleic acid sequence with that of Geo thermodenitrificans; and mdh gene shares 99% identity of the nucleic acid sequence with that of Bacillus thermodenitrificans-m. The identity of the nucleic acid sequence of recN gene with that of Geo-thermodenitrifican is 99%. Among all of the bacteria compared with G1788, the result of Geobacillus thermodenitrificans shows the highest identity. The results above-mentioned indicates that G1788 shares the closest relationship with Geobacillus thermodenitrificans, such that it can be recognized that G1788 belongs to Geobacillus thermodenitrificans.

### 3) Screening of Bacterial Strains

(1) The steps of strains screening. Use the strains of Applicants' lab and strains in the 40 aqueous samples from strata of Dagang Oil Field as the original strains for screening. (1) Primary screening: by being cultured and screened with agar medium plate at 73° C., then being incubated with mineral nutrition medium used crude oil as carbon resource, being shaken in oil bath at 73° C., then being experienced emulsifying for dispersing, the best dispersed strains are obtained. (2) Re-screening: by conducting the experiments of emulsifying for dispersing and the experiments of decreasing viscosity and freezing point to choose the strains that dispersed and emulsified best as well as viscosity and freezing point decreasing most, the strain of G1788 is obtained. (3) The strain of G1788 is inoculated to the mineral nutrition medium supplemented with liquid wax at 73° C. several times to be domesticated and propagated.

The Experiments of Emulsifying for Dispersing:

The 250 ml Erlenmeyer Flasks are filled respectively with 100 ml mineral nutrition medium and 2 g dehydrated crude oil, disinfected at 121° C. for 30 min, and the inoculation amount is 10%. The Erlenmeyer Flasks are then airproofed at 73° C., incubated in oil bath for 7 days. They are then cooled under room temperature to observe the emulsification and decentralization result with naked eyes. It may be measured with eight grades from “4−” to “4+” (4+ stands for the best). The best state is that the oil does not hang on the wall, and the droplets of the oil are all small and homogeneous. When the culture medium forms a homogeneous suspension and looks like Chinese ink after being shaken, it is considered to be the best grade (“4+”), the pH value and the surface tension of the liquid phase is determined. The crude oil degradation rate of the oil phase is determined (mineral nutrition medium g/L; Na₂HPO₄ 0.06; KH₂PO₄ 0.02; NaNO₃ 0.2; CaCl₂ 0.0001; FeSO₄ 0.001; MgSO₄ 0.03; yeast powder 0.05; sucrose 0.1; pH value is 7.2). Basic medium; liquid wax or crude oil is added to mineral nutrition medium, used to emulsifying for dispersing experiments etc.

The experiments of decreasing viscosity and freezing point:

The 250 ml Erlenmeyer Flasks are filled respectively with 30 ml mineral nutrition medium and 40 g dehydrated crude oil, disinfected at 121° C. for 30 min, and the inoculation amount of 30 ml. The Erlenmeyer Flasks are then airproofed at 73° C., incubated in oil bath for 7 days. The Erlenmeyer Flasks are put into the refrigerator remaining at 4° C. after being cooled under room temperature, they are taken out and dehydrated three times after the oil phase is frozen completely. The viscosity of the oil phase is then determined at 50° C., and the freezing point is determined according to the method of oil analysis and estimate (oil industry press 2000, Page 34-35).

According to the results of the experiments above, G1788 is selected to be the best one to degrade oil.

The strain of Geobacillus thermodenitrificans G1788 obtained by screening has already been deposited in China General Microbiological Culture Collection Center, its conserving number is CGMCC-1228.

(2) The optimizing of strain culture medium (the “percentage” in the culture medium) means the mass (g) of solute in every 100 ml solution. For example, glucose 1% means that there is 1 g glucose contained in 100 ml solution.

Reagents: Gravy culture medium (%): Beef cream 0.4; Peptone 1; NaCl 0.5.
Nutritional Agar medium (5): Gravy culture medium +1.8% Agars

Method: Bacterial Colonies Counting on Nutritional Agar Plate

I. Single Factor test

TABLE 3
Selection of carbon resource, nitrogen resource and growth factor

| Bacterial | Carbon | Nitrogen | With | Without |
|---|---|---|---|---|
| Strain | resource | resource | growth factor | growth factor |
| G1788 | Glucose | NaNO₃ | 6.8 × 10⁶ | 9.6 × 10⁵ |
| | | (NH₄)₂SO₄ | 8.6 × 10⁵ | 7.4 × 10⁵ |
| | | NH₄Cl | 1.11 × 10⁴ | 6.8 × 10³ |
| | | NH₄H₂PO₄ | 9.55 × 10⁴ | 5.7 × 10⁴ |
| | Sucrose | NaNO₃ | 1.58 × 10⁷ | 2.88 × 10⁶ |
| | | (NH₄)₂SO₄ | 2.0 × 10⁶ | 1.9 × 10⁵ |
| G1788 | | NH₄Cl | 2.57 × 10⁵ | 2.4 × 10⁵ |
| | | NH₄H₂PO₄ | 2.22 × 10⁵ | 2.01 × 10⁵ |
| | | NaNO₃ | | 1.99 × 10⁶ |
| | | (NH₄)₂SO₄ | | 5.82 × 10⁴ |
| | | NH₄Cl | | 7.10 × 10⁴ |
| | | NH₄ H₂PO₄ | | 4.21 × 10³ |

(The culture medium is added in liquid wax by ratio of 2% in addition. The mineral nutrition ingredients are the same as that in the basic medium. Amount of glucose, sucrose, or starch is 0.1%, and amount of NaNO₃ is 0.2%. Other nitrogen resources are added in by the same molar concentration as NaNO₃). The results indicate that it grows best in the condition of using sucrose as the carbon resource, using NaNO₃ as the nitrogen resource with the growth factor.

II. Selection of the mineral nutrition ingredients is shown in Table 4.

TABLE 4
Selection of the mineral nutrition ingredients.

| Bacterial | The mineral | Amount of |
|---|---|---|
| Strain | nutrition absent | bacteria |
| G1788 | CaCl₂ | 3.88 × 10⁶ |
| | FeSO₄ | 6.75 × 10⁴ |
| | MgSO₄ | 5.87 × 10⁶ |
| | Na₂HPO₄ | 3.33 × 10⁴ |
| | KH₂PO₄ | 6.2 × 10⁴ |
| | None of the above | 1.43 × 10⁷ |

The concentrations of the mineral nutrition ingredients above-mentioned are the same as that of the mineral nutrition culture medium. The results indicate that the bacteria grow well when all the five kinds of mineral nutrition ingredients in the table are adequate.

III. Selection of the optimal pH is shown in Table 5

TABLE 5
Selection of the optimal pH

| Bacterial | | Amount of |
|---|---|---|
| Strain | pH | bacteria |
| G1788 | 5.6 | 2.7 × 10⁶ |
| | 6.4 | 3.5 × 10⁴ |
| | 7.2 | 6.4 × 10⁶ |
| G1788 | 8.0 | 3.2 × 10⁶ |
| | 8.8 | 8.1 × 10⁵ |

The results indicate that the condition of pH 7.2 is the optimal condition for culturing the bacteria.

IV. Referring to Table 6, where the determination of the optimal culture condition is shown.

TABLE 6
Results of the orthogonal experimental design

| | Column No. | | | | |
|---|---|---|---|---|---|
| | 1 | 2 | 4 | 7 | |
| | Factor | | | | |
| | | Sucrose | | Mineral | |
| | NaNO₃ (%) | (B) | Yeast Powder (%) | Nutrition | Bacteria |
| Assay No. | A | B | C | D | Concentration |
| 1 | 0.2 | 0.1 | 0.05 | D1 | 7.2 × 10⁷ |
| 2 | 0.2 | 0.1 | 0.10 | D2 | 3.89 × 10⁷ |
| 3 | 0.2 | 0.2 | 0.05 | D2 | 2.75 × 10⁶ |
| 4 | 0.2 | 0.2 | 0.10 | D1 | 3.5 × 10⁶ |
| 5 | 0.4 | 0.1 | 0.05 | D2 | 1.25 × 10⁸ |
| 6 | 0.4 | 0.1 | 0.10 | D1 | 2.59 × 10⁷ |
| 7 | 0.4 | 0.2 | 0.05 | D1 | 8.2 × 10⁶ |
| 8 | 0.4 | 0.2 | 0.10 | D2 | 2.31 × 10⁷ |
| K₁ | 11.72 × 10⁷ | 26.18 × 10⁷ | 20.80 × 10⁷ | 10.96 × 10⁷ | |
| K₂ | 18.22 × 10⁷ | 3.76 × 10⁷ | 9.14 × 10⁷ | 18.98 × 10⁷ | |

The four factors are arranged into two levels to conduct orthogonal experiment. The table is designed according to the L₈ (27) form, the experiment project is formed from column 1, 2, 4 and 7.

NaNO₃ (%) A1 . . . 0.2; A2 . . . 0.4; Sucrose (%) B1 . . . 0.1; B2 . . . 0.2;

Yeast powder (%) C1 . . . 0.05; C2 . . . 0.1 Mineral nutrition . . . D1 (same as that of mineral nutrition medium); D2 (two times more D1)

The statistical analysis indicates that B and C are the two major factors affecting the results. So based upon the above experiments, B1, C1, A2, D2 are selected as the optimal culture medium, namely NaNO₃ 0.4%, Sucrose 0.1%, Yeast powder 0.05% and mineral nutrition concentration is two times more than that of basic medium.

V. The results of the validation experiment are shown in Table 7.

TABLE 7
Results of the validation experiment.

| | | Final | |
|---|---|---|---|
| | | concentration | |
| Bacterial | Initial | Basic medium | |
| Strain | Concentration | w/liquid wax | Optimized medium |
| G1788 | 1.07 × 10⁵ | 4.12 × 10⁶ | 1.4 × 10⁸ |

The results indicate that the optimized medium is significantly better than the basic medium with liquid wax.

### 4). Advantageous Effects of the Present Invention

(1) Accommodating to the strata environment.

I. The strains grow well in the strata water at 73° C. The strains are cultured in the basic mediums with liquid wax confected from strata water and distilled water respectively at 73° C. for 4 days, the respective growth status is compared with each other and the results are shown in Table 8.

TABLE 8
The strains accommodate well to the strata water

| Bacterial | | Initial | Final |
|---|---|---|---|
| strain | Culture medium | concentration | concentration |
| G1788 | Distilled water | 1.01 × 10⁵ | 6.6 × 10⁶ |
| | Strata water | 1.06 × 10⁵ | 6.9 × 10⁶ |

The strata water is provided by Dagand Oil Field, the ion type and content of which are (mg/L) NA⁺K⁺ 6075; Mg² 87; Ca²⁺ 298; Cl⁻ 9874; SO4²⁻ 37; HCO₃⁻ 419. The strata water is highly mineralized, and contains endogenesis microorganisms, being very different from the distilled water. However, the above-mentioned results indicate that the strata water is the distilled water. However, the above-mentioned results indicate that the strata water and its endogenesis microorganisms do not affect the growth of the strain of G1788, which can grow normally in the strata water, and accommodate well.

II. The experiments of emulsifying for dispersing in the strata water culture condition.

Measurement of the degradation rate of the crude oil: 2.00 g dehydrated crude oil is weighed up accurately and used for the experiments of emulsifying for dispersing. The ferment liquid is filtrated, and all of the undegrated crude oil is gathered by hexane, and diluted properly. Measure the value of OD at 254 nm. The strains which have not been shaken are used as the blank comparison to calculate the remaining amount of crude oil, then calculate the degradation rate of the crude oil. The results are shown in Table 9.

TABLE 9
Degradation rate of the crude oil

| | | | | Degradation rate |
|---|---|---|---|---|
| Bacterial | | Dispersing | Surface | of the |
| strain | Culture | results | tension | crude oil % |
| G1788 | Distilled water | 3− | 65.0 | 7.98 |
| | (sterilized) | | | |
| | Distilled water | 2+ | 71.6 | 3.79 |
| | (unsterilized) | | | |
| | Strata water | 4+ | 72.2 | 4.33 |
| | (sterilized) | | | |
| | Strata water | 4+ | 65.2 | 12.1 |
| | (unsterilized) | | | |

It can be seen from the above table that the strain of G1788 has more positive effect on the crude oil when it is cultured in (sterilized) strata water than in (unsterilized) strata water, which indicate that the strata water and its endogenesis microorganisms do not inhibit the growth of thermophyla bacteria and its ability of degrading the crude oil. The degradation rate of the crude oil is much more higher when the strain of G1788 is cultured in strata water (unsterilized) than in the distilled water, which indicate that the strain of G1788 accommodate the strata water of the oil field and other microbial environment, it can grow normally in the oil reservoirs, and degrade crude oil efficiently.

(2) Heat-resistance

I. The growth temperature range of the bacterial strain is shown in Table 10 (inoculating according to the method of streaking plate with nutritional agar plate, culturing at different temperatures and observing the growth status).

TABLE 10
The growth temperature range of the bacterial strain

| Bacterial | Temperature | | | | | | | | | |
|---|---|---|---|---|---|---|---|---|---|---|
| Strain | 37° C. | 40° C. | 42° C. | 45° C. | 55° C. | 65° C. | 73° C. | 75° C. | 78° C. | 80° C. |
| G1788 | − | − | − | + | + | + | + | + | + | − |

The growth temperature range of the bacterial strain is 45° C.˜78° C.

II. The growth of bacterial strain at different temperatures are shown in table 11 (Counting colonies on the nutritional agar plate, drawing the growth curves of 60° C., 65° C. and 73° C.).

TABLE 11
Growth under different temperatures

| Bacterial | | Time (h) | | | | | | | |
|---|---|---|---|---|---|---|---|---|---|
| strain | Temperature | 0 | 12 | 24 | 36 | 48 | 60 | 72 | 84 |
| G1788 | 60° C. | 10^{6.5} | 10^{6.7} | 10^{7.1} | 10^{7.3} | 10^{7.3} | 10^{6.9} | 10^{6.7} | 10^{6.8} |
| | 65° C. | 10^{6.5} | 10^{7.0} | 10^{8.4} | 10^{8.5} | 10^{8.1} | 10^{7.4} | 10^{7.0} | 10^{7.0} |
| | 73° C. | 10^{6.5} | 10^{6.3} | 10^{6.6} | 10^{6.8} | 10^{7.2} | 10^{7.1} | 10^{6.8} | 10^{6.4} |

The concentration of the bacteria can reach 10⁸/ml after 24 hours incubation. The incubation condition may be: culturing for 24 hours with selected optimal culture medium at 65° C.

III. The temperature growth curve is shown in Table 12 (Counting colonies on the nutritional agar plate, after two-day incubation at the following temperature, measuring the concentration of the bacteria, and drawing the temperature growth curve).

TABLE 12
Temperature growth curve

| Bacterial | Temperature | | | | | | |
|---|---|---|---|---|---|---|---|
| strain | 50° C. | 55° C. | 60° C. | 65° C. | 70° C. | 73° C. | 78° C. |
| G1788 | 10^{3.7} | 10^{4.0} | 10^{6.6} | 10^{8.2} | 10^{7.8} | 10^{6.0} | 10^{3.5} |

The optimal growth temperature range is 60° C.˜73° C.

IV. The effects of freezing and dissolving on the growth of the bacteria. The assayed performance data of low-temperature-resistance is shown in Table 13. (Counting colonies on the nutritional agar plate, freezing the bacteria in refrigerators (−20° C.) for several periods shown in the following table respectively, measuring the bacteria concentration).

TABLE 13
Assayed performance data of low-temperature-resistance

| Bacterial | Time | | | | |
|---|---|---|---|---|---|
| strain | 0 day | 1 day | 3 days | 4 days | 5 days |
| G1788 | 8.9 × 10⁶ | 8.0 × 10⁶ | 8.96 × 10⁶ | 9.18 × 10⁶ | 9.31 × 10⁶ |

After freezing for a certain time, the bacterial concentration of G1788 is generally unchanged. The strain shows good low-temperature-resistance character, which is of great significance in winter quarry application.

(3) Growing with crude oil as the sole carbon source, referring to FIG. 5.

Adding to 2% glucose or crude oil to the BSM medium, the growth condition is assessed by CFU counting method. G1788 is able to grow well in the culture medium which uses crude oil as the sole carbon source, the lag stage of which is longer slightly and the growth quantity of which in the late logarithm stage is slightly less, as compared with the sample which grows in the medium added glucose. (The BSM medium containing 2.44 g KH₂PO₄, 5.57 g Na₂HPO₄, 2 g NH₄CL, 0.2 MgCL₂, 0.01 CaCl₂, 0.001 FeCl₃.6H₂O, and 0.04 g MnCl₂.4H₂O per liter, pH=7.2).

(4) Degrading the crude oil

I. The effects on crude oil by the bacterial stain

The emulsifying for dispersing experiment and viscosity-decreasing experiment are performed, using crude oil as the sole carbon source. The samples are incubated respectively for 5, 10, 15, 20 days in oil bath at 73° C. The samples are divided into the test group (added G1788) and the controlled group (cultured in the conditions same as that of test group, but without any bacteria).

A. The pH change of the ferment liquid is shown in table 14.

TABLE 14
the pH change of the ferment liquid

| | Days | | | | |
|---|---|---|---|---|---|
| Samples | 0 day | 5 days | 10 days | 15 days | 20 days |
| G1788 | 7.8 | 7.2 | 7.0 | 6.9 | 7.0 |
| Controlled | 8.0 | 7.9 | 7.9 | 7.8 | 7.8 |

B. The surface tension change of the ferment liquid is shown in table 15.

TABLE 15
The surface tension change of the ferment liquid.

| | Days | | | | |
|---|---|---|---|---|---|
| Samples | 0 day | 5 days | 10 days | 15 days | 20 days |
| G1788 | 76 | 73 | 71 | 70 | 71 |
| Controlled | 76 | 72 | 72.5 | 73 | 75 |

C. Degradation of the crude oil (the degradation rate %) is shown in table 16.

TABLE 16
The degradation rate of the crude oil

| | Days | | | | |
|---|---|---|---|---|---|
| Samples | 0 day | 5 days | 10 days | 15 days | 20 days |
| G1788 (%) | 0 | 13 | 21 | 32 | 39 |
| Controlled (%) | 0 | 2 | 7 | 12 | 13 |

B. The viscosity-decreasing effect (the viscosity-decreasing rate) is shown in table 17.

TABLE 17
The viscosity-decreasing rate of the crude oil

| | Days | | | | |
|---|---|---|---|---|---|
| Samples | 0 day | 5 days | 10 days | 15 days | 20 days |
| G1788 (%) | 7.8 | 7.2 | 7.0 | 6.9 | 7.0 |
| Controlled (%) | 8.0 | 7.9 | 7.9 | 7.8 | 7.8 |

V. The freezing point of the crude oil (° C.) is shown in table 18.

TABLE 18
The freezing point of the crude oil (° C.)

| | Days | | | | |
|---|---|---|---|---|---|
| Samples | 0 day | 5 days | 10 days | 15 days | 20 days |
| G1788 | 46° C. | 45.5° C. | 46° C. | 44.5° C. | 43.5° C. |
| Controlled | 46° C. | 46° C. | 46° C. | 45.5° C. | 45.5° C. |

The results shown above indicate that G1788 significantly increases the degradation rate of the crude oil, and decreases the surface tension and freezing point of the crude oil, thus significantly decreases the viscosity of the crude oil. Both degradation rate and fluidity are increased after the crude oil being processed by G1788. So it is much easier to exploit, therefore, the application of G1788 can increase the recovery rate of the crude oil.

II. The degradation of the crude oil by the strain is measured by spectrometry, and the results are shown in table 19.

A. Adding 2.00 crude oil to the high temperature mineral nutrition medium, and G1788 is incubated in it, and its growth status is observed. G1788 is able to grow well in the medium. The high temperature mineral nutrition medium used for this experiment contains 0.34% of KH₂PO₄, 0.15% of Na₂HPO₄, 0.4% of (NH₄)₂SO₄, 0.07% of MgSO₄ and 0.05% of yeast extract, pH=7.2

B. The samples are fermented for 120 h and 168 h respectively to perform emulsifying for dispersing experiment. The flasks are held still for 30 min once the time runs over and the emulsified status is observed. The oil is mostly dispersed homogeneously in water phase, comparing to the controlled with only a little residue of the oil flowing on the water phase surface. The residue of the sample fermented for 168 h is less than that of the sample fermented for 120 h.

C. The crude oil is diluted with hexane, and its full-wavelength absorption is is measured. It is seen that there is an obvious absorb peak at 250 nm, and amount of the oil is correlate to A_{250 nm}, R²=0.9975, y=0.0137x. The emulsifying for dispersing experiment is kept running for 1-5 days. Hexane is used to extract the crude oil in the fermenting culture medium. The extracted sample is diluted properly to measure A_{250 nm}, and amount of the oil is determined by using the standard curve.

TABLE 19
The degradation of the crude oil

| Days | Controlled | 1 | 2 | 3 | 4 | 5 |
|---|---|---|---|---|---|---|
| Oil amount (g) | 1.8 | 1.7 | 1.6 | 1.2 | 0.7 | .038 |

The 2.00 g of oil in the medium decrease with the growth and metabolism of G1788 in five days, with the oil degraded to 0.38 g on the fifth day. So it can be seen that G1788 obviously accelerate the degradation of crude oil. (Note: spectrophotometry is a rough method. It is affected by many factors, therefore it is usually used as a qualitative analysis, and is not used as a quantitative analysis. However in this experiment, the results remarkably indicate that the G1788 has a good degradation effect on the oil).

III. Analysis of the degradation of the oil by using gas chromatography

A, Apparatus and Reagents

A, Apparatus:

KS_{—}501 digital orbital shaker;

Agilent Technologies 6890N gas chromatograph;

Gas chromatograph column: varian cp7542, length: 10 m, inner diameter: 0.53 mm, thickness: 0.17 μm;

MaxTerm: 450° C.

B, Analysis Condition

Inlet: temperature is 400° C., stress is 3.6 Kpa, and the feed-in is prepared through cold column;

The temperature of the stove: using programmed two-step method to increase the temperature of the stove. The initial temperature is 60° C., speed is 5° C./min; the terminal temperature is 250° C., speed is 4° C./min; and the terminal temperature is 380° C., keeping constant for 10 min.

Detector: the hydrogen flame detector, the temperature of detecting condition is 400° C., assistance is N₂ (the flow rate is 20 ml/min), the flow rate of H₂ is 40.0 ml/min, and the flow rate of air is 460.0 ml/min.

IV. The Culturing of Bacteria and the Treatment on Samples

A. The Culture Medium Used.

a. LB Medium:

Peptone 1%, Yeast powder 0.5%, NaCl 1%, pH=7.0;

b. Liquid Wax Inducing Culture Medium:

Na₂HPO₄ 0.06%, KH₂PO₄ 0.02%, NaNO₃ 0.4%, CaCl₂ 0.001%, FeSO₄ 0.001%, MgSO₄ 0.003%, Yeast powder 0.1%, Sucrose 0.5%, pH is 7.2, Liquid wax 2%;

c. High Temperature Mineral Nutrition Medium

KH₂PO₄ 0.34%, Na₂HPO₄ 0.15%, (NH₄)₂SO₄ 0.4%, MgSO₄ 0.07%, Yeast powder 0.05%, pH is 7.2;

d. Ordinary Agar Slant or Plate Culture Medium:

NaCl 0.5%, Peptone 1%, Beef cream 0.4%, Agar 3%, pH is 7.2-7.5;

The culture mediums above-mentioned are all disinfected at 121° C. for 30 min before used.

B. The Culturing of the Bacterial Strains.

a. Three rings of the bacterial strains in the glycerin freezing tubes are selected to inoculate into a 50 mL LB flask, which is then shaken for culturing for 24 h at 65° C. under 120 rpm;

b. The samples are inoculated to 50 mL liquid wax inducing medium, shaking for culturing for 24 h;

c. The samples are then inoculated to 100 mL liquid wax inducing medium, shaking for culturing for 24 h under same condition;

d. The samples are inoculated to 100 mL high temperature mineral nutrition medium, shaking for culturing for 24 h under same condition. The samples are taken out at different fermenting time, and are measured to determine the degradation rate of the crude oil caused by this bacterial body;

e. The quantities of the inoculation are all 10%.

C. The treatment on samples The ferment liquid is poured into a 250 mL separatory funnel, and 100 mL of hexane is divided into three portions and added into separatory funnel in three times, every time, it is poured into the shake flask which is then blocked tightly with a plastic plug, and oscillated on the level shaking bed at 200 r/min for 30˜60 min, then transferred into separatory funnel, oscillated rotationally, and put aside for at least overnight.

The upper clear liquid in the separatory funnel is carefully taken out with micro-sampler, then centrifuged twice at 12000 r/min, and preserved in the refrigerator of −20° C.

The treatment on the original sample of the crude oil: 2 grams of oil is weighed up accurately, diluted with 100 mL of hexane, oscillated to mix, centrifuged twice at 12,000 r/min and preserved in the refrigerator of −20° C.

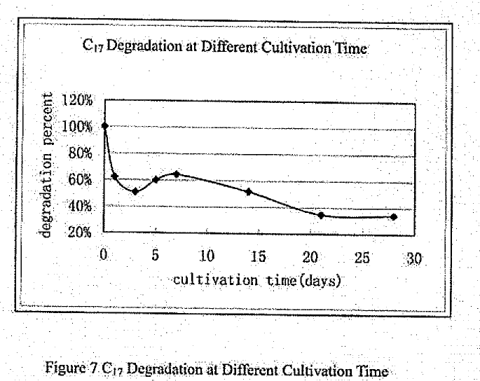

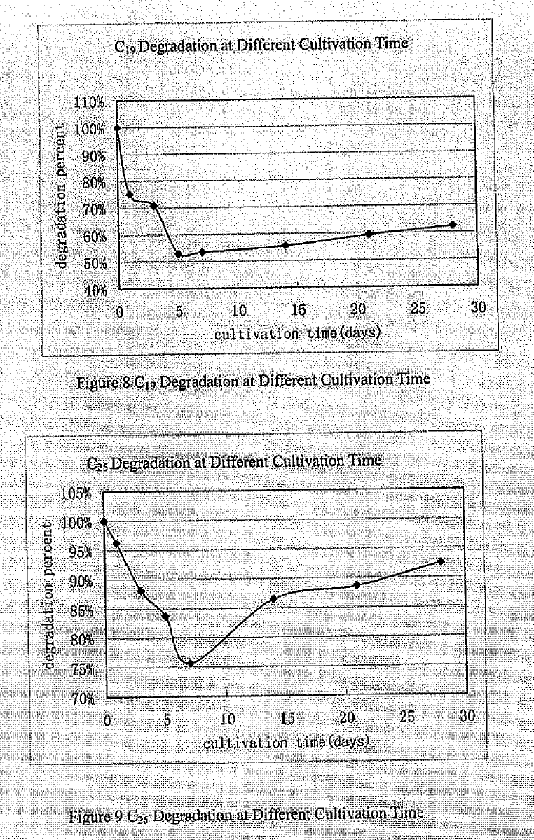

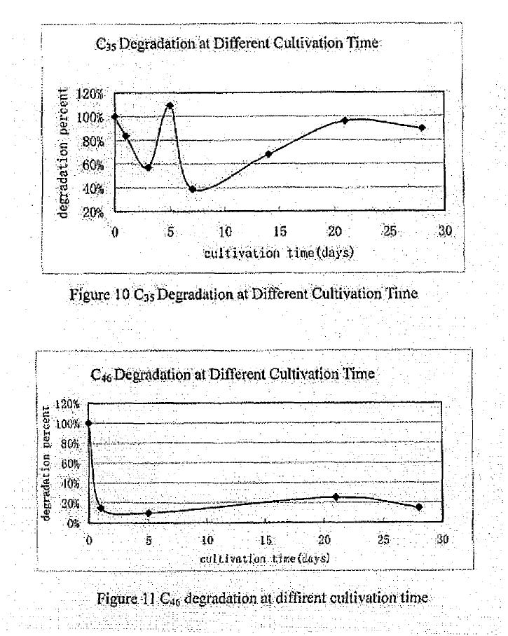

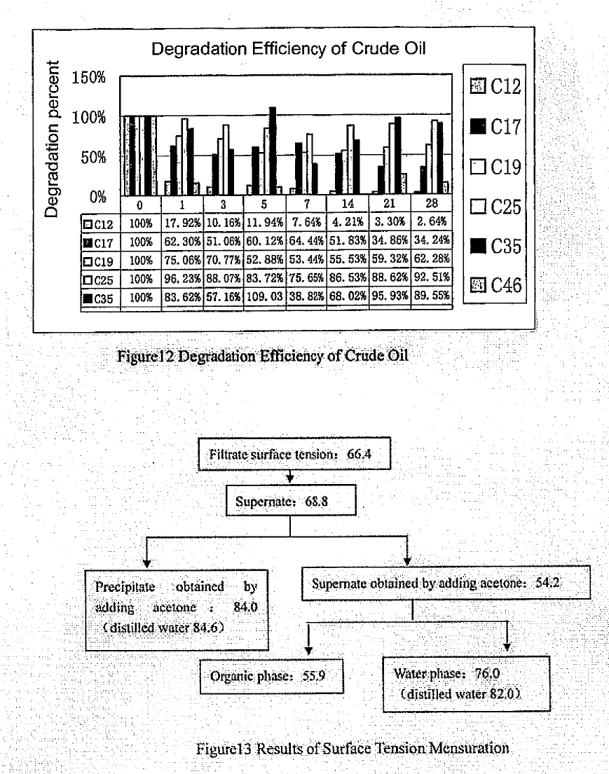

D. Determining the oil degradation status during respective fermentation period with GC shows that oil concentration of the original sample of the crude oil is 2%. The results are shown in FIGS. 6 to 12.

(5) The comparison between the degradations of alkane, as shown in FIGS. 6 to 12.

It can be seen from the above figures that the degradation of the linear alkanes generally appeared in the first three days, for C12, C17, and C19. The degradation rate changes most greatly in the first five days, for C25. An obvious degradation phenomenon appears in the first three days, but the amount gradually increases in the several days later, even more than that of the first day (1.073%). This may be attributed to that the high molecular weight linear alkanes are degraded to approximate carbons alkanes, causing an increasing of the amount of the alkanes. The increasing of C35 on the fifth day is caused by the degradation of the higher carbon alkane. The degradation result of C46 is very good. The results indicate that the bacterial strain of the present invention can degrade alkanes of up to 46 carbon chain hydrocarbons in the degradation of the oil. In this experiment, the oil degradation on respective different fermentation time is determined by inner calibration method. To is reduce the system tolerance of the experiment, it is decided to compare the degradation status with that of the first day based on the later one to obtain the degradation rate, due to the phenomenon of cell emulsified oil is obvious. The degradation effect will be more obvious if based on the undegraded original crude oil.

Accordingly, the strain of Geobacillus thermodenitrificans G1788 provided in present invention belongs to Geobacillus thermophily. It has the capability of heat-resistance, using crude oil as the sole carbon source, growing well in oil reservoir environment, therefore it can improve the quality of the crude oil through the growth and metabolism using the crude oil as the solo carbon source, degrade alkane much more, and can increase the fluidity of the crude oil, consequently, and enhance the performance of oil recovery efficiently.

Another advantageous effect of the strain of Geobacillus thermodenitrificans G1788 provided in present invention is that, the strain has the petroleum-degrading capability. The growth and metabolism of the strain in certain petroleum-containing substances will perform a good purification effect on these substances by eliminating petroleum. At present, due to the development of the petroleum industry, the petroleum pollution on people's living environment generated by recovery, transportation, and production of petroleum, many petroleum-contaminated substances caused very serious environmental pollution, such as petroleum-containing sewage, the leakage of crude oil in the environment, and so on. By using the strain screened by the present invention, the petroleum in these substances may be degraded, the contaminants may be purified, and the using of the strain may play an important role in environmental protection, particularly in the field of process technology on oil pollution. It is of significant value.

(6) Application of the strain of the present invention in industrial production such as fermentation requiring thermal-stable enzymes.

Researchers have shown that the enzymes isolated and purified from the thermophilic bacteria have good thermal stability, which can be applied to industrial production, scientific research and so on.

Almost all of the enzymes from thermophilic bacteria are thermo-stable. The thermal stability of enzymes from thermophilic bacteria is determined by the internal structure of the enzyme protein molecule. Researches show that the primary structure of enzyme itself plays an important role in its thermal stability. The changes of individual amino acids in some key regions of the primary structure will cause the changes in higher order structure of enzyme, and will slightly increase the number of hydrogen bond, ionic bond or hydrophobic bond in the structure of enzyme protein, thereby improve the thermal stability of the whole molecule. The thermal stability of the enzyme synthesized by thermophilic bacteria may be changed either by changing the temperature or by changing the configuration of enzyme protein existed.

The enzymes separated from thermophilic bacteria have some excellent biological characters, such as thermal stability, and resistance to chemical and physical denaturing agent, organic solvents, extreme pH, and other unfavorable factors. These characters may not only be used as the basis for designing and modifying enzymes, but also be of important application value for industrial production.

There are many advantages in using the thermophile enzymes generated by thermophile bacteria as the bio-catalyzer:

I. Reducing the cost, and enhancing the stability of the enzymes so they can be purified and packaged for transporting at room temperature while maintaining activity as long as possible.

II. Accelerating the kinetic reaction.

III. Reducing the requirements for the cooling system while reacting, thus reducing energy consumption, this will lower costs as well as reduce environmental pollution caused by the cooling process.

IV. Under the thermophile-enzyme-catalyzed reaction condition, the impurity strains appear barely, thus contamination is reduced and the purity of the product is improved. As a result, the purity of the product is improved and the purification process is simplified.

V. Since the biochemical reaction process can be carried out at high temperature, the solubility and availability of insoluble materials are increased, and the viscosity of organic compounds is reduced, facilitating the diffuse and mix.

Due to the above reasons, the thermophile strains of the invention, as well as the thermophile enzymes generated by it both have a broad prospect of application in food, chemistry, pharmaceutical industry and environment protection. Similarly, the enzymes related to the strains, such as the enzyme catalyzed degradation of long-chain alkanes, the surfactant secretion also have broad application prospects. Thermophilic enzymes are encoded and expressed by the corresponding gene of the thermophilic bacteria, in order to reduce production costs, a method commonly used is that, by using genetic engineering technology, the gene of the thermophile enzymes is extracted from the strain, then loaded into a plasmid. The plasmid containing the gene is then transferred into certain bacteria to establish an efficient expression system for producing such thermophilic enzymes. The enzymes which catalyze the degradation of petroleum hydrocarbon and the surfactant secretion, and is encoded and synthesized by the corresponding gene expression or coding and synthesis, may also be made available by large-scale production using the same technology of genetic engineering. The technology of genetic engineering has already been a conventional method in biotechnology industries. Ail of the genes, protein enzymes and other useful substances related to the characteristics of strains of the present invention can be extracted and applied using genetic engineering technology.

(7) The screened strain of the present invention has the ability to produce surfactant secretion.

The strain of the present invention has the ability to produce materials decreasing surface tension or surface active substances, experiments of emulsifying for dispersing are carried out by using the bacteria, the medium liquid used in the experiments of emulsifying for dispersing is used to perform surfactant analysis.

I. The determination of the extraction method of surfactant is shown in FIG. 14.

II. Analysis of the surfactant:

Quantitative determination of the raw product: The dry products from rotational film evaporation are dissolved by chloroform, then the solution is transferred to a vial having been weighed, and the vial is weighed.

Qualitative Analysis: TLC (Thin Layer Chromatography) Two-step expansion is used. Petroleum ether:ether:acetic acid (80:20:1, in volume) are expanded at first. Stop expansion when the distance between the forefront and the top forward is about 1 cm, then use chloroform:methanol:water (65:15:2, in volume) for re-expansion. Stop re-expansion when the forefront passes half length of the plate. The plate is then observed under UV, colored by iodine vapor, phenol-sulfuric acid, 0.5% ninhydrin acetone solution, and ammonium molybdate-perchloric acid solution.

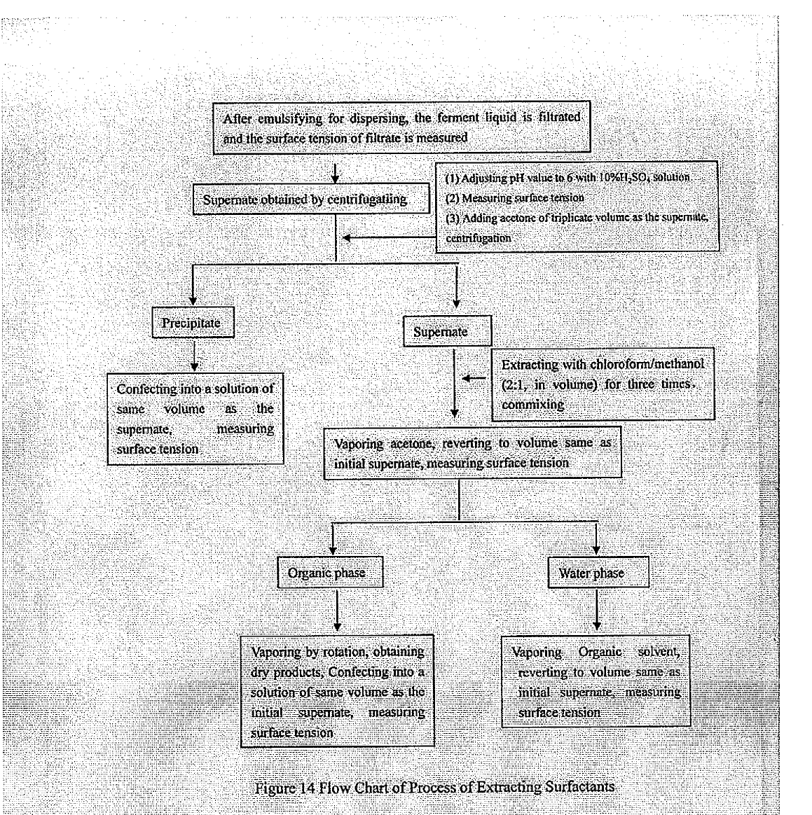

III. Surface Tension results are shown in FIG. 13.

After the organic solvent extraction, the organic phase contributes most to the surface tension, which suggests that the components of the surfactants are mainly lipid compounds.

IV. Qualitative and quantitative results are shown in table 18.

TABLE 20
Yield of the surfactant

| | 5 days | | 10 days | | 15 days | | 20 days | |
|---|---|---|---|---|---|---|---|---|
| | G1788 | Controlled | G1788 | Controlled | G1788 | Controlled | G1788 | Controlled |
| Weight of | 11.88 | 7.14 | 21.48 | 15.12 | 36.72 | 24.72 | 13.02 | 10.44 |
| the raw | | | | | | | | |
| product | | | | | | | | |
| (mg/ml) | | | | | | | | |
| Yield | 0.198 | 0.119 | 0.358 | 0.252 | 0.612 | 0.412 | 0.217 | 0.174 |
| rate (g/L) | | | | | | | | |

Raw products of the surfactant is a mixture of several substances with various polarity: (1) non-polar substances or neutral materials are near the frontier, which are phosphate (2) the spots in the central of the expansion agent react positively to iodine vapor, phenol-sulfuric acid, showing that it is glycolipid compounds. The spots appearing on the fifth day and the 15th day react positively to ninhydrin, showing that they are lipopeptide materials. There are still some materials with strong polarity, which do not exist in the controlled group. It has been reported that the composition of crude oil is very complicated. It contains a number of active ingredients, i.e. the materials with surface activity itself, the active ingredients are mostly long-chain fatty acid, oxygenated compounds such as alcohol or hydroxybenzene, and other oxygenated compounds such as organic acid. Besides, the ways of microbe effecting on crude oil are various, intermediate products are rich, thus many different types of surfactants are generated. The above-mentioned results suggest that the G1788 can significantly increase the content of surfactants, and reduce the surface tension of the crude oil, thereby increasing the fluidity of the crude oil.

(8) The strain of the present invention has the characteristic of decreasing the viscosity of the oil; therefore it can be used in the transportation of the oil.

The emulsifying for dispersing experiment and viscosity-decreasing experiment are performed, using crude oil as the sole carbon source. The samples are incubated respectively for 5, 10, 15, 20 days in oil bath at 73° C. The samples are divided into the test group (added G1788} and the controlled group (cultured in the conditions same as that of test group, but without any bacteria). The viscosity-decreasing rates of the crude oil are shown in Table 21 (same as table 17).

TABLE 21

| | Days | | | | |
|---|---|---|---|---|---|
| Group | O d | 5 d | 10 d | 15 d | 20 d |
| G1788 (%) | 0 | 4 | 9 | 17 | 27 |
| controlled: (%) | 0 | 2 | 3 | 4 | 7 |

The data in the table show that G1788 can significantly decrease the viscosity of the crude oil. The conceivable explanation is that the bacteria may generate a lot of metabolic substances (such as surfactants), and that the degradation of long-chain petroleum hydrocarbons has effectively improved the fluidity of the crude oil.

In the exploitation and transportation of the oil, the high viscosity of the oil makes it difficult to flow, so more production processes and more energy consumption are needed. Therefore, people have developed a variety of ways to decrease oil viscosity. For instance, cracking wave radiation can decrease viscosity. Since the strain of the invention 10 has very good effect on decreasing oil viscosity, the strain or the materials extracted from the strain can effectively decrease viscosity of the crude oil, if they are applied to the oil exploitation and petroleum transportation industry, the mining rate and transportation efficiency will be effectively improved.

Preferred embodiments of the invention are described above. The description is not intended to restrict the invention in any way, and any modification, equivalents and adaptation of the above embodiments based on the spirit of the invention are still within the scope of the present invention.

1. A strain of Geobacillus thermodenitrificans which is deposited in China General Microbiological Culture Collection Center of China Committee of Culture Collection for Microorganisms, having the number of CGMCC-1228.

2. A method for screening the strain of Geobacillus thermodenitrificans according to claim 1, wherein the strains isolated from the aqueous samples of the strata of oil field is used as the original strains for screening, and the strain is achieved after experiencing primary screening and re-screening, inoculation, domestication, and propagation.

3. A method for screening the strain of Geobacillus thermodenitrificans according to claim 2, wherein the primary screening is carried out by culturing and screening the samples with agar medium plate at 73° C., then the samples obtained are incubated with mineral nutrition medium used crude oil as carbon source, shaken in oil bath at 73° C., and emulsified for dispersing, then the best dispersed strains are obtained; the re-screening is conducted in such a way that: in experiments of emulsifying for dispersing and experiments of decreasing viscosity and freezing point, select the strains which were dispersed and emulsified best and the viscosity and freezing point thereof decreased most, therefore the best strain is obtained; then the best strain is inoculated with the mineral nutrition medium supplemented with liquid was at 73° C. several times to be domesticated and propagated.

4. An optimal culture medium for the strain of Geobacillus thermodenitrificans according to claim 1 which includes carbon, nitrogen and mineral nutrition sources, and yeast powder, wherein the carbon source is glucose, sucrose or starch, the nitrogen source is NaNO₃, mineral source is FeSO₄, MgSO₄, Na₂HPO₄, and K₂HPO₄, the pH of the medium is 6.5-7.5.

5. An optimal culture medium for the strain of Geobacillus thermodenitrificans according to claim 4, wherein said carbon source is sucrose of which the amount used is 0.1%; said nitrogen source is NaNO₃ of which the amount used is 0.1%-0.4%; the amount of said yeast powder used is 0.05%, the amount of said mineral nutrition is 1.5-2 times more than that of basic medium.

6. The uses of the strain of Geobacillus thermodenitrificans according to claim 1 in oil exploration, oil hydrocarbon degradation, cleansing of material containing oil, and oil transportation field.

7. The uses of the strain of Geobacillus thermodenitrificans according to claim 1 in industrial production that requiring thermo-stability enzymes.

8. The uses of the strain of Geobacillus thermodenitrificans according to claim 1 are in industry of biosurfactant preparation.

9. The uses of the strain of Geobacillus thermodenitrificans according to claim 6, wherein said materials containing oil is the sewage contaminated by oil, etc.

10. The uses of the strain of Geobacillus thermodenitrificans according to claim 7, wherein said industrial production requiring thermo-stable enzyme is fermentation industrial production.
